(19) United States
(12) Patent Application Publication
(10) Pub. No.: US 2026/0061242 A1
(43) Pub. Date: Mar. 5, 2026

(54) BIOREMEDIATION OF PERCHLORATES VIA BACTERIUM GENE INSERTION

(71) Applicant: Daniel Park, Wesley Chapel, FL (US)

(72) Inventor: Daniel Park, Wesley Chapel, FL (US)

(21) Appl. No.: 18/815,970

(22) Filed: Aug. 27, 2024

(51) Int. Cl. A62D 3/02; A62D 101/49; C12N 1/20; C12N 9/02; C12N 15/09; C12R 1/19

(52) U.S. Cl. CPC A62D 3/02; C12N 1/205; C12N 9/0069; C12N 15/09; C12Y 113/11049; C12Y 197/01; A62D 2101/49; C12R 2001/19

(57) ABSTRACT

Aspects of the disclosure include the manufacture of non-naturally occurring bacteria for perchlorate bioremediation. An exemplary method includes determining a source bacteria having one or more source genes which code for perchlorate reduction and chlorite dismutase and determining a target bacteria that does not naturally include the source genes. A first sequence of deoxyribonucleic acid (DNA) is identified in the source genes that codes, in the source bacteria, for an ordered set of amino acids whose linear sequence results in a protein which breaks down perchlorates and a second sequence of DNA is determined which, if inserted into the target bacteria, would allow the target bacteria to code for the ordered set of amino acids. A gene package is built by replacing the first sequence of DNA with the second sequence of DNA and the gene package is inserted into the target bacteria, thereby forming a non-naturally occurring modified bacteria.

The present disclosure relates to environmental pollution and bioremediation measures, and particularly to the use of gene insertion in bacterium for the bioremediation of perchlorates.

Bioremediation refers to a class of environmental cleanup approaches that utilize living organisms, typically microbes, to break down or neutralize hazardous substances and pollutants in an environment. Bioremediation can harness the natural metabolic capabilities of these organisms to transform (metabolize) pollutants into less harmful or non-toxic substances. Bioremediation has gained significant attention in recent decades as a cost-effective and environmentally friendly alternative to traditional chemical and physical remediation methods.

Perchlorates refer to a class of compounds that contain the perchlorate ion ClO₄⁻ and are persistent environmental contaminants that have become a significant concern due to their widespread presence and potential health and environmental impacts. Perchlorates include an inorganic anion that forms a salt with various cations. Perchlorates are highly soluble in water and stable under typical environmental conditions, which contributes to a widespread persistence in the environment. The primary sources of perchlorate contamination include military and defense operations, as perchlorate is used in solid rocket propellants, explosives, and pyrotechnics, industrial applications, as perchlorates are used in the production of airbag inflators, fireworks, and certain types of fertilizers, and natural occurrences, as perchlorates can form naturally in the atmosphere and precipitate in arid regions.

Perchlorate contamination is particularly problematic because perchlorates easily dissolve in and pollute groundwater, with the potential to affect drinking water sources for large populations. Perchlorates can also accumulate in food crops irrigated with contaminated water. Perchlorates also interfere with iodide uptake in the thyroid gland, potentially disrupting thyroid hormone production. This is especially concerning for pregnant women and developing fetuses, as thyroid hormones are crucial for normal development. Perchlorates have been detected in human breast milk, raising concerns about infant exposure. Interestingly, perchlorate has even been detected in Martian soil at concentrations of 0.5 to 1 percent, which is significantly higher than typical terrestrial levels. This discovery has implications for potential human exploration and colonization of Mars, as these levels would be toxic to humans and plants.

### SUMMARY

In one exemplary embodiment a method includes determining a source bacteria having one or more source genes which code for perchlorate reduction and chlorite dismutase and determining a target bacteria that does not naturally include the source genes. A first sequence of deoxyribonucleic acid (DNA) is identified in the source genes that codes, in the source bacteria, for an ordered set of amino acids whose linear sequence results in a protein which breaks down perchlorates and a second sequence of DNA is determined which, if inserted into the target bacteria, would allow the target bacteria to code for the ordered set of amino acids. A gene package is built by replacing the first sequence of DNA with the second sequence of DNA and the gene package is inserted into the target bacteria, thereby forming a non-naturally occurring modified bacteria. The method includes applying the modified bacteria to a substrate having perchlorates for perchlorate bioremediation.

In addition to one or more of the features described herein, in some embodiments, the source bacteria includes a bacteria of the genus Dechloromonas, the genus Dechlorosoma, or the genus Azospira.

In some embodiments, the target bacteria includes Escherichia coli (E. coli).

In some embodiments, the one or more source genes includes a perchlorate reductase A gene (pcrA), a perchlorate reductase B gene (pcrB), a perchlorate reductase C gene (pcrC), a perchlorate reductase D gene (pcrD), and a chlorite dismutase gene (cld).

In some embodiments, determining the second sequence of DNA which, if inserted into the target bacteria, would allow the target bacteria to code for the ordered set of amino acids includes determining a first codon frequency for one or more codons which code for amino acids in the ordered set of amino acids in the source bacteria, determining a second codon frequency for the one or more codons in the target bacteria, and modifying the first sequence of DNA by replacing one or more codons which code for the amino acids in the ordered set of amino acids with replacement codons which code for the same respective amino acids but have a higher codon frequency in the target bacteria. In some embodiments, the resulting sequence includes the second sequence of DNA.

In some embodiments, the method includes applying a growth medium for the modified bacteria to the substrate.

In some embodiments, the method includes, responsive to a concentration of a targeted compound in the substrate falling below a predetermined threshold, purging the substrate of the modified bacteria.

In some embodiments, the targeted compound includes one of perchlorate, chlorate, or chlorite.

In some embodiments, the method includes periodically or continuously sampling the substrate to determine a current concentration of the targeted compound.

In some embodiments, the method includes growing a culture of the modified bacteria prior to substrate application.

In another exemplary embodiment a non-naturally occurring Escherichia coli includes a gene package having a sequence of DNA that codes for an ordered set of amino acids. In some embodiments, the ordered set of amino acids contain a linear sequence that codes for a protein which breaks down perchlorates.

In some embodiments, the gene package is derived by determining a source bacteria having one or more source genes which code for perchlorate reduction and chlorite dismutase, identifying a first sequence of DNA in the one or more source genes that codes, in the source bacteria, for the ordered set of amino acids, determining a second sequence of DNA which, if inserted into naturally occurring Escherichia coli, would allow the resulting non-naturally occurring Escherichia coli to code for the ordered set of amino acids, building the gene package by replacing the first sequence of DNA in the one or more source genes with the second sequence of DNA, and inserting the gene package into naturally occurring Escherichia coli, thereby forming a non-naturally occurring Escherichia coli.

In some embodiments, the source bacteria includes a bacteria of the genus Dechloromonas. In some embodiments, the source bacteria includes a bacteria of the genus Dechlorosoma. In some embodiments, the source bacteria includes a bacteria of the genus Azospira.

In some embodiments, the one or more source genes includes a perchlorate reductase A gene (pcrA). In some embodiments, the one or more source genes includes a perchlorate reductase B gene (pcrB). In some embodiments, the one or more source genes includes a perchlorate reductase C gene (pcrC). In some embodiments, the one or more source genes includes a perchlorate reductase D gene (pcrD). In some embodiments, the one or more source genes includes a chlorite dismutase gene (cld).

In some embodiments, determining the second sequence of DNA which, if inserted into naturally occurring Escherichia coli, would allow the resulting non-naturally occurring Escherichia coli to code for the ordered set of amino acids includes determining a first codon frequency for one or more codons which code for amino acids in the ordered set of amino acids in the source bacteria, determining a second codon frequency for the one or more codons in the target bacteria, and modifying the first sequence of DNA by replacing one or more codons which code for the amino acids in the ordered set of amino acids with replacement codons which code for the same respective amino acids but have a higher codon frequency in the target bacteria. In some embodiments, the resulting sequence is the second sequence of DNA.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings.

FIG. 1 is a process for modifying bacteria using gene insertion for the bioremediation of perchlorates; and

FIG. 2 is a flowchart in accordance with one or more embodiments.

### DETAILED DESCRIPTION

The following description is merely exemplary in nature and is not intended to limit the present disclosure, its application or uses. It should be understood that throughout the drawings, corresponding reference numerals indicate like or corresponding parts and features.

Certain bacteria, particularly those belonging to the genera Dechloromonas and Dechlorosoma, have evolved the ability to use perchlorate as an electron acceptor in their metabolism, effectively breaking down the contaminate. This capability makes these bacterium excellent candidates for perchlorate bioremediation efforts. The key to their effectiveness lies in a specific set of genes that code for enzyme perchlorate reductase. In particular, these bacteria have a perchlorate reductase enzyme complex encoded by a cluster of four genes: pcrA, pcrB, pcrC, and pcrD (collectively, the “pcrABCD genes”). These genes work together to produce the enzyme complex that catalyzes the reduction of perchlorate. Without wishing to be bound by theory, and allowing for some overlap in enzymatic pathways, pcrA and pcrB form the core catalytic subunits of perchlorate reductase and are similar in structure and function to the α- and β-subunits of other enzymes in the dimethyl sulfoxide (DMSO) reductase family, such as nitrate reductase, selenate reductase, and chlorate reductase. More particularly, pcrA is the α-subunit of perchlorate reductase and contains the active site where perchlorate reduction occurs and is the largest subunit for catalytic activity. The PcrA subunit has been shown to have a relatively high affinity for perchlorate (Km=6 μM) and exhibits substrate inhibition, distinguishing it from similar enzymes like respiratory nitrate reductase. PcrB is the B-subunit of perchlorate reductase and likely plays a role in electron transfer within the enzyme complex. The pcrC gene encodes a protein similar to a c-type cytochrome and suggests that PcrC is involved in electron transfer, potentially shuttling electrons from the bacteria cell's electron transport chain to the catalytic subunits (PcrA and PcrB) of perchlorate reductase. The pcrD gene product shows similarity to molybdenum chaperone proteins found in other DMSO reductase family members and indicates that PcrD likely plays a role in the assembly of the perchlorate reductase enzyme complex, particularly in incorporating the molybdenum cofactor into the active site of Car.

In any case, the perchlorate reduction pathway in bacteria such as Dechloromonas and Dechlorosoma proceeds in a stepwise manner where perchlorate (ClO₄⁻) is initially reduced to chlorate (ClO₃⁻), which is then further reduced to chlorite (ClO₂⁻). Since chlorite is cytotoxic, the chlorite is further broken down by a separate protein called chlorite dismutase, coded for by a gene called Cld, into oxygen and chloride (this process is typically referred to as chlorite dismutation). In short, this pathway allows these types of bacteria to effectively break down perchlorate into less harmful products, with the final step producing relatively harmless chloride ions and oxygen.

Unfortunately, while bacterial perchlorate reduction via bacteria such as Dechloromonas and Dechlorosoma shows great promise, there are several limitations and challenges inherent to those bacteria genera which natively limit widespread application. Perhaps most importantly, Dechloromonas and Dechlorosoma are anaerobic bacteria, meaning that these bacteria thrive in oxygen-free environments. More specifically, the expression of the pcrA gene in Dechloromonas agitata occurs only under anaerobic (per) chlorate-reducing conditions. The presence of oxygen completely inhibits natural pcrA expression, regardless of the presence of perchlorate, chlorate, or nitrate, suggesting that naturally evolved perchlorate reductase systems are part of the anaerobic respiratory pathway. As a result, these types of bacteria (e.g., Dechloromonas and Dechlorosoma) are severely restricted in the types of environments in which they can be readily applied for perchlorate bioremediation. Scaling is also a challenge, as Dechloromonas and Dechlorosoma grow in anaerobic environments and cannot readily culture to industrial scale in air and/or aerated surface soils.

This disclosure introduces a process for gene insertion in bacterium for the bioremediation of perchlorates. Specifically, the pcrABCD genes and the Cld gene are inserted into nonpathogenic Escherichia coli (E. coli) to allow the resulting genetically modified (non-naturally occurring) E. coli to break down perchlorates. This approach presents a novel method of bioremediation not only for areas on Earth contaminated with perchlorates but also for treating soils on Mars and other environments in preparation for future space missions. This method would make bioremediation more accessible, as E. coli is more readily available than the specific types of bacteria that normally degrade perchlorates. In addition, E. coli requires less specialized growth conditions than the bacteria naturally containing the pcrABCD and Cld genes, which are typically anaerobic.

Potential users of the methods described herein include everyday consumers seeking to reduce perchlorate pollution in their water, farmers seeking an easy method of remediating perchlorate pollution in their crops, ecologists looking to degrade perchlorates in a polluted environment, as well as future researchers on Mars and other systems looking to transform soil into a nontoxic substance(s) facilitating human colonization. Once the genetically modified E. coli is cultured, application is straightforward, as users only need to simply release the engineered bacteria in an area contaminated with perchlorates. The modified E. coli will then degrade the perchlorates naturally without need for specialized equipment or chemicals. Thus, users would not need to rely on currently available processes which are relatively inaccessible, such as, for example, electrodialysis, conductive carbon nanotube nanocomposites, etc.

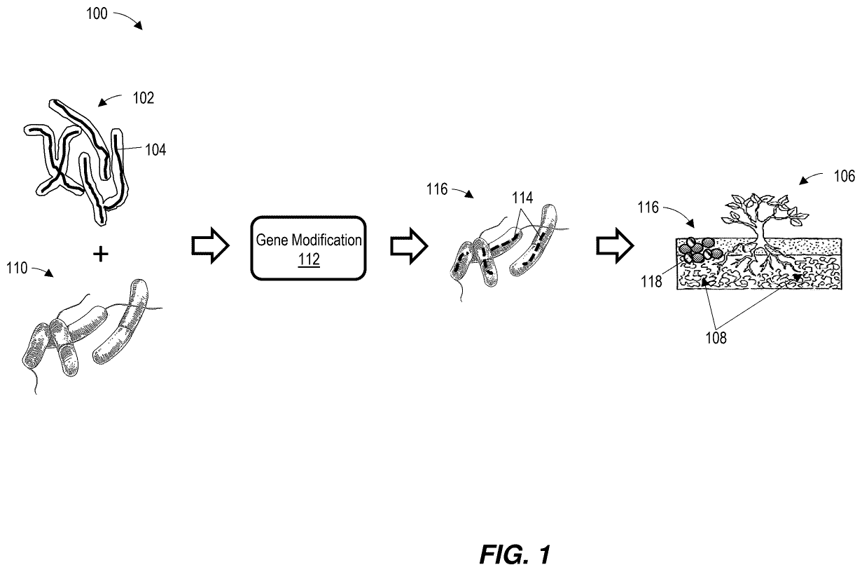

FIG. 1 illustrates a process 100 for modifying bacteria using gene insertion for the bioremediation of perchlorates in accordance with one or more embodiments. As shown in FIG. 1, a source bacteria 102 is identified and/or otherwise determined. In some embodiments, the source bacteria 102 is a bacteria having one or more source genes 104. In some embodiments, the source genes 104 code for perchlorate reduction and/or chlorite dismutase. The source bacteria 102 is not meant to be particularly limited, but can include, for example, bacteria of the genus Dechloromonas, bacteria of the genus Dechlorosoma, or bacteria of the genus Azospira. Other bacteria having source genes which code for perchlorate reduction and/or chlorite dismutase are known and are within the contemplated scope of this disclosure. In some embodiments, the source bacteria 102 is an anaerobic bacteria. In some embodiments, the source bacteria 102 is a bacteria having a first growth rate in a predetermined substrate 106 (environment, soil, air, river, lake, ocean, stream, landfill, field, etc.). In some embodiments, the predetermined substrate 106 is a substrate containing perchlorates 108 either naturally, via contamination, or from a combination of natural and nonnatural causes. In some embodiments, the predetermined substrate 106 is a target for the bioremediation of perchlorates.

The one or more source genes 104 can include, for example, one or more of a perchlorate reductase A gene (also referred to as the perchlorate reductase alpha subunit precursor gene, or pcrA), a perchlorate reductase B gene (pcrB), a perchlorate reductase C gene (pcrC), a perchlorate reductase D gene (pcrD), and a chlorite dismutase gene (cld). In some embodiments, the one or more source genes 104 can include exactly one of pcrA, pcrB, pcrC, and pcrD. In some embodiments, the one or more source genes 104 can include any combination of pcrA, pcrB, pcrC, and pcrD. In some embodiments, the one or more source genes 104 can include pcrA, pcrB, pcrC, and pcrD (collectively, “pcrABCD”). In some embodiments, the one or more source genes 104 can include cld. In some embodiments, the one or more source genes 104 can include pcrABCD and cld.

As further shown in FIG. 1, the process 100 includes determining and/or otherwise identifying a target bacteria 110 that does not naturally include the one or more source genes 104 of the source bacteria 102. In some embodiments, the target bacteria 110 is a naturally occurring bacteria. In some embodiments, the target bacteria 110 is a nonpathogenic bacteria. In some embodiments, the target bacteria 110, in contrast to the source bacteria 102, is an aerobic bacteria. In some embodiments, the target bacteria 110 is a bacteria having a second growth rate in the predetermined substrate 106 (environment, soil, air, river, lake, ocean, stream, landfill, field, etc.) that is relatively higher than the first growth rate of the source bacteria 102 in the predetermined substrate 106. In some embodiments, the target bacteria 110 is nonpathogenic Escherichia coli (E. coli), although other target bacteria are possible and within the contemplated scope of this disclosure. Other target bacteria can include any bacteria capable of being genetically modified using a same or similar procedure as described below with respect to E. coli. Other target bacteria can include aerobic bacteria and/or bacteria having a higher growth rate in the predetermined substrate 106 than the source bacteria 102.

As further shown in FIG. 1, the process 100 includes gene modification 112. In some embodiments, gene modification 112 includes identifying a first sequence of deoxyribonucleic acid (DNA) in the one or more source genes 104 that codes, in the source bacteria 102, for an ordered set of amino acids whose linear sequence results in a protein which breaks down perchlorates. In some embodiments, gene modification 112 includes determining a second sequence of DNA which, if inserted into the target bacteria 110, would allow the target bacteria 110 to code for the ordered set of amino acids. In some embodiments, gene modification 112 includes building a gene package 114 by replacing the first sequence of DNA in the one or more source genes 104 with the second sequence of DNA. In some embodiments, gene modification 112 includes inserting the gene package 114 into the target bacteria 110, thereby forming a non-naturally occurring modified bacteria 116 (or simply “modified bacteria 116”). In some embodiments, the modified bacteria 116 is a modified E. coli. In some embodiments, the modified E. coli is modified for perchlorate bioremediation. Gene modification 112 is discussed in greater detail below.

As further shown in FIG. 1, the process 100 includes applying the modified bacteria 116 to the predetermined substrate 106 containing perchlorates 108. Advantageously, the modified bacteria 116 will degrade the perchlorates 108, thereby enabling a bioremediation of the predetermined substrate 106. In some embodiments, a reduction pathway utilized by the modified bacteria 116 proceeds from perchlorates 108 to chlorate, which is then reduced to chlorite. This process is mediated via a protein coded for by pcrA, pcrB, pcrC, pcrD, and/or pcrABCD. Since the resulting chlorite is cytotoxic, in some embodiments, the reduction pathway utilized by the modified bacteria 116 further includes breaking down chlorites into oxygen and chloride via a separate protein, coded for by cld, called chlorite dismutase. In this manner, the modified bacteria 116 can be applied to the predetermined substrate 106 for perchlorate bioremediation.

In some embodiments, the process 100 includes the application of a growth medium 118 to the modified bacteria 116 and/or to the predetermined substrate 106. Growth mediums are known, and are not meant to be particularly limited. In some embodiments, growth medium 118 is selected to promote the growth rate of the modified bacteria 116 and/or otherwise to support the modified bacteria 116. In some embodiments, the process 100 includes growing a culture of the modified bacteria 116 prior to applying the modified bacteria 116 to the predetermined substate 106. In this manner, the modified bacteria 116 can be grown to scale in controlled conditions prior to bioremediation efforts.

In some embodiments, the process 100 includes periodically or continuously sampling (not separately indicated) the predetermined substrate 106 to determine a current concentration of a targeted compound (e.g., perchlorates 108). In some embodiments, the process 100 includes, responsive to a concentration of the targeted compound in the predetermined substrate 106 falling below a predetermined threshold, purging the predetermined substrate 106 of the modified bacteria 116. In some embodiments, the targeted compound includes one or more of perchlorate, chlorate, or chlorite. In some embodiments, purging the predetermined substrate 106 of the modified bacteria 116 includes destroying the modified bacteria 116. Processes for destroying bacteria are known and are not meant to be particularly limited.

### Gene Modification and Codon Optimization

In some embodiments, a modified bacteria 116 is created by modifying a target bacteria 110 using gene modification 112 for perchlorate bioremediation, as described previously with respect to FIG. 1. Gene modification 112 will now be described in greater detail.

In some embodiments, gene modification 112 includes determining a second sequence of DNA which, if inserted into the target bacteria 110, would allow the target bacteria 110 to code for the ordered set of amino acids previously identified with respect to the source bacteria 102 and one or more source genes 104 (specifically, the ordered set of amino acids whose linear sequence results in a protein which breaks down perchlorates).

In some embodiments, determining the second sequence of DNA includes determining a first codon frequency for one or more codons which code for amino acids in the ordered set of amino acids in the source bacteria 102, determining a second codon frequency for the one or more codons in the target bacteria 110. In some embodiments, determining the second sequence of DNA further includes modifying the first sequence of DNA by replacing one or more codons which code for the amino acids in the ordered set of amino acids with replacement codons which code for the same respective amino acids but have a higher codon frequency in the target bacteria 110. In some embodiments, the resulting sequence is the second sequence of DNA.

To illustrate this process (referred to herein as “codon optimization”), consider a scenario in which bacteria of the genus Azospira are the source bacteria 102 and E. coli is the target bacteria 110. Codon frequencies for Azospira and E. coli are known and thus, codon frequencies can be compared (refer to Tables 1 and 2 below).

TABLE 1
Azospira Codon Frequencies

| | | | | | | | |
|---|---|---|---|---|---|---|---|
| TTT | 14.57 | TCT | 9.00 | TAT | 13.72 | TGT | 4.29 |
| (Phe) | | (Ser) | | (Tyr) | | (Cys) | |
| TTC | 31.72 | TCC | 7.29 | TAC | 22.72 | TGC | 12.86 |
| (Phe) | | (Ser) | | (Tyr) | | (Cys) | |
| TTA | 3.86 | TCA | 8.57 | TAA | 1.29 | TGA | 0.00 |
| (Leu) | | (Ser) | | (Ter) | | (Ter) | |
| TTG | 16.72 | TCG | 14.57 | TAG | 0.86 | TGG | 19.72 |
| (Leu) | | (Ser) | | (Ter) | | (Trp) | |
| CTT | 13.72 | CCT | 15.43 | CAT | 11.57 | CGT | 13.72 |
| (Leu) | | (Pro) | | (His) | | (Arg) | |
| CTC | 11.57 | CCC | 12.43 | CAC | 12.43 | CGC | 18.43 |
| (Leu) | | (Pro) | | (His) | | (Arg) | |
| CTA | 8.14 | CCA | 9.86 | CAA | 12.86 | CGA | 7.29 |
| (Leu) | | (Pro) | | (Gln) | | (Arg) | |
| CTG | 26.58 | CCG | 24.43 | CAG | 25.72 | CGG | 6.00 |
| (Leu) | | (Pro) | | (Gln) | | (Arg) | |
| ATT | 19.72 | ACT | 12.86 | AAT | 20.57 | AGT | 6.43 |
| (Ile) | | (Thr) | | (Asn) | | (Ser) | |
| ATC | 21.86 | ACC | 25.72 | AAC | 22.72 | AGC | 8.14 |
| (Ile) | | (Thr) | | (Asn) | | (Ser) | |
| ATA | 4.29 | ACA | 7.29 | AAA | 32.58 | AGA | 2.14 |
| (Ile) | | (Thr) | | (Lys) | | (Arg) | |
| ATG | 29.15 | ACG | 14.14 | AAG | 38.58 | AGG | 0.86 |
| (Met) | | (Thr) | | (Lys) | | (Arg) | |
| GTT | 11.57 | GCT | 17.57 | GAT | 29.15 | GGT | 13.29 |
| (Val) | | (Ala) | | (Asp) | | (Gly) | |
| GTC | 21.43 | GCC | 24 | GAC | 27.86 | GGC | 31.72 |
| (Val) | | (Ala) | | (Asp) | | (Gly) | |
| GTA | 8.14 | GCA | 17.57 | GAA | 28.29 | GGA | 12.86 |
| (Val) | | (Ala) | | (Glu) | | (Gly) | |
| GTG | 20.15 | GCG | 18.86 | GAG | 28.72 | GGG | 9.86 |
| (Val) | | (Ala) | | (Glu) | | (Gly) | |

TABLE 2
E. Coli Codon Frequencies

| | | | | | | | |
|---|---|---|---|---|---|---|---|
| TTT | 22.18 | TCT | 8.37 | TAT | 15.9 | TGT | 5.09 |
| (Phe) | | (Ser) | | (Tyr) | | (Cys) | |
| TTC | 16.59 | TCC | 8.57 | TAC | 12.16 | TGC | 6.37 |
| (Phe) | | (Ser) | | (Tyr) | | (Cys) | |
| TTA | 13.76 | TCA | 6.91 | TAA | 2.05 | TGA | 0.92 |
| (Leu) | | (Ser) | | (Ter) | | (Ter) | |
| TTG | 13.63 | TCG | 8.88 | TAG | 0.21 | TGG | 15.24 |
| (Leu) | | (Ser) | | (Ter) | | (Trp) | |
| CTT | 10.98 | CCT | 6.92 | CAT | 12.73 | CGT | 21.17 |
| (Leu) | | (Pro) | | (His) | | (Arg) | |
| CTC | 11.15 | CCC | 5.40 | CAC | 9.66 | CGC | 22.2 |
| (Leu) | | (Pro) | | (His) | | (Arg) | |
| CTA | 3.87 | CCA | 8.40 | CAA | 15.29 | CGA | 3.50 |
| (Leu) | | (Pro) | | (Gln) | | (Arg) | |
| CTG | 53.48 | CCG | 23.57 | CAG | 28.92 | CGG | 5.13 |
| (Leu) | | (Pro) | | (Gln) | | (Arg) | |
| ATT | 30.58 | ACT | 8.88 | AAT | 17.28 | AGT | 8.58 |
| (Ile) | | (Thr) | | (Asn) | | (Ser) | |
| ATC | 25.29 | ACC | 23.44 | AAC | 21.57 | AGC | 16.06 |
| (Ile) | | (Thr) | | (Asn) | | (Ser) | |
| ATA | 4.03 | ACA | 6.73 | AAA | 33.66 | AGA | 1.82 |
| (Ile) | | (Thr) | | (Lys) | | (Arg) | |
| ATG | 28.07 | ACG | 14.4 | AAG | 10.14 | AGG | 1.00 |
| (Met) | | (Thr) | | (Lys) | | (Arg) | |
| GTT | 18.34 | GCT | 15.4 | GAT | 32.03 | GGT | 25 |
| (Val) | | (Ala) | | (Asp) | | (Gly) | |
| GTC | 15.25 | GCC | 25.79 | GAC | 19.1 | GGC | 29.96 |
| (Val) | | (Ala) | | (Asp) | | (Gly) | |
| GTA | 10.98 | GCA | 20.28 | GAA | 39.89 | GGA | 7.74 |
| (Val) | | (Ala) | | (Glu) | | (Gly) | |
| GTG | 26.54 | GCG | 34.26 | GAG | 17.74 | GGG | 10.95 |
| (Val) | | (Ala) | | (Glu) | | (Gly) | |

Now, referring to Table 1 and Table 2, the listed codon frequencies denote an approximate estimate for how often the respective codon appears per 1000 codons. These codon frequencies can be converted into relative percentages trivially. For example, E. coli codon TTT has a codon frequency of 22.18 while TTC has a codon frequency of 16.59, and both code for phenylalanine (Phe). Adding the results for all Phe codons (in this case, two codons) gives a total frequency of 38.77. Now, on a relative basis, TTT has a frequency of 57.2 percent (22.18/38.77*100) and TTC has a frequency of 42.8 percent (16.59/38.77). Observe that the codon frequencies for phenylalanine (Phe) are different between Azospira and E. Coli. Specifically, the Azospira codon frequencies for Phe are 14.57 for TTT and 31.72 for TTC (refer to Table 1), while the E. coli codon frequencies for Phe are 22.18 for TTT and 16.59 for TTC (refer to Table 2). Thus, from this comparison it can be observed that Azospira prefers the TTC codon for coding Phe, while E. coli prefers the TTT codon for coding Phe.

Now, continuing with this example, an initial sequence of DNA (e.g., the first sequence of DNA of the source bacteria 102 of FIG. 1) can be modified by replacing one or more codons which code for the amino acids in the ordered set of amino acids with replacement codons which code for the same respective amino acids but have a higher codon frequency in the target bacteria 110. Specifically, in this example, TTC codons in the first sequence of DNA can be replaced with TTT codons. This process can then be repeated to replace codons for all amino acids coded in the first sequence of DNA, thereby building a second sequence of DNA which is codon-optimized for the target bacteria 110. A modified bacteria 116 can then be created from the target bacteria 110 by inserting a gene package including the second sequence of DNA into the target bacteria 110 (refer to FIG. 1). Gene insertion techniques are known, such as CRISPR for precise editing, electroporation to create temporary pores in the cell membrane, allowing DNA entry, and other gene-editing tools, and are not meant to be particularly limited.

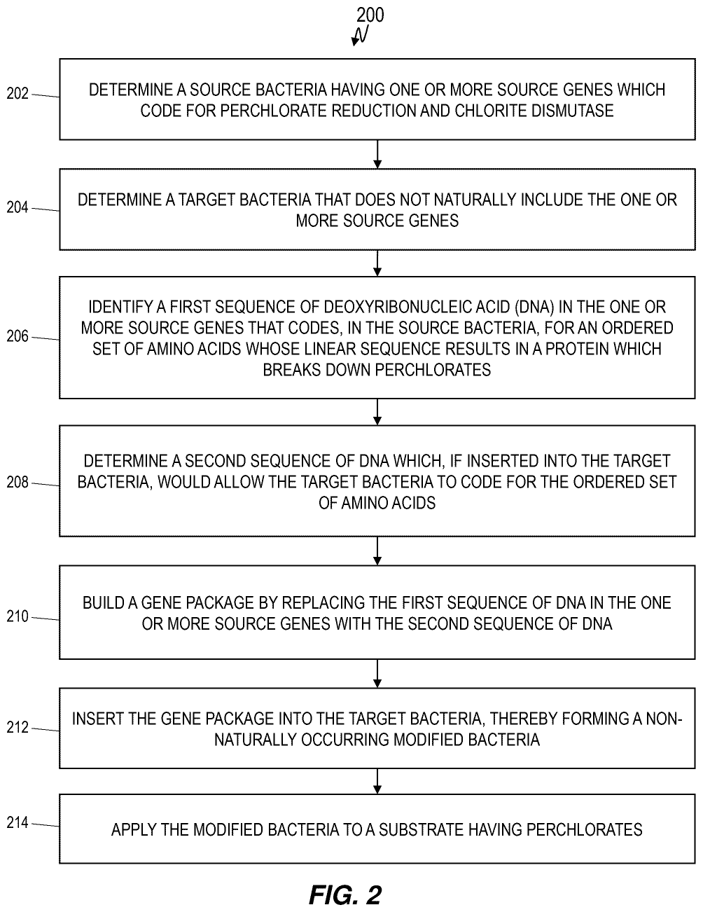

Referring now to FIG. 2, a flowchart 200 for perchlorate bioremediation is generally shown according to an embodiment. The flowchart 200 may include additional steps not depicted in FIG. 2. Although depicted in a particular order, the blocks depicted in FIG. 2 can, in some embodiments, be rearranged, subdivided, and/or combined.

At block 202, the method includes determining a source bacteria having one or more source genes which code for perchlorate reduction and chlorite dismutase.

At block 204, the method includes determining a target bacteria that does not naturally include the one or more source genes.

At block 206, the method includes identifying a first sequence of deoxyribonucleic acid (DNA) in the one or more source genes that codes, in the source bacteria, for an ordered set of amino acids whose linear sequence results in a protein which breaks down perchlorates.

At block 208, the method includes determining a second sequence of DNA which, if inserted into the target bacteria, would allow the target bacteria to code for the ordered set of amino acids.

At block 210, the method includes building a gene package by replacing the first sequence of DNA in the one or more source genes with the second sequence of DNA.

At block 212, the method includes inserting the gene package into the target bacteria, thereby forming a non-naturally occurring modified bacteria.

At block 214, the method includes applying the modified bacteria to a substrate having perchlorates for perchlorate bioremediation.

In addition to one or more of the features described herein, in some embodiments, the source bacteria includes a bacteria of the genus Dechloromonas, the genus Dechlorosoma, or the genus Azospira.

In some embodiments, the target bacteria includes Escherichia coli (E. coli).

In some embodiments, the one or more source genes includes a perchlorate reductase A gene (pcrA), a perchlorate reductase B gene (pcrB), a perchlorate reductase C gene (pcrC), a perchlorate reductase D gene (pcrD), and a chlorite dismutase gene (cld).

In some embodiments, determining the second sequence of DNA which, if inserted into the target bacteria, would allow the target bacteria to code for the ordered set of amino acids includes determining a first codon frequency for one or more codons which code for amino acids in the ordered set of amino acids in the source bacteria, determining a second codon frequency for the one or more codons in the target bacteria, and modifying the first sequence of DNA by replacing one or more codons which code for the amino acids in the ordered set of amino acids with replacement codons which code for the same respective amino acids but have a higher codon frequency in the target bacteria. In some embodiments, the resulting sequence includes the second sequence of DNA.

In some embodiments, the method includes applying a growth medium for the modified bacteria to the substrate.

In some embodiments, the method includes, responsive to a concentration of a targeted compound in the substrate falling below a predetermined threshold, purging the substrate of the modified bacteria.

In some embodiments, the targeted compound includes one of perchlorate, chlorate, or chlorite.

In some embodiments, the method includes periodically or continuously sampling the substrate to determine a current concentration of the targeted compound.

In some embodiments, the method includes growing a culture of the modified bacteria prior to substrate application.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

Additionally, as used in this disclosure, phrases of the form “at least one of an A, a B, or a C,” “at least one of A, B, and C,” and the like, should be interpreted to select at least one from the group that comprises “A, B, and C.” Unless explicitly stated otherwise in connection with a particular instance in this disclosure, this manner of phrasing does not mean “at least one of A, at least one of B, and at least one of C.” As used in this disclosure, the example “at least one of an A, a B, or a C,” would cover any of the following selections: {A}, {B}, {C}, {A, B}, {A, C}, {B, C}, and {A, B, C}.

When an element such as a layer, film, region, or substrate is referred to as being “on” another element, it can be directly on the other element or intervening elements may also be present. In contrast, when an element is referred to as being “directly on” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this disclosure belongs.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. A method for perchlorate bioremediation, the method comprising:
determining a source bacteria comprising one or more source genes which code for perchlorate reduction and chlorite dismutase;
determining a target bacteria that does not naturally include the one or more source genes;
identifying a first sequence of deoxyribonucleic acid (DNA) in the one or more source genes that codes, in the source bacteria, for an ordered set of amino acids whose linear sequence results in a protein which breaks down perchlorates;
determining a second sequence of DNA which, if inserted into the target bacteria, would allow the target bacteria to code for the ordered set of amino acids;
building a gene package by replacing the first sequence of DNA in the one or more source genes with the second sequence of DNA;
inserting the gene package into the target bacteria, thereby forming a non-naturally occurring modified bacteria; and
applying the modified bacteria to a substrate comprising perchlorate for perchlorate bioremediation.

2. The method of claim 1, wherein the source bacteria comprises a bacteria of the genus Dechloromonas, the genus Dechlorosoma, or the genus Azospira.

3. The method of claim 1, wherein the target bacteria comprises Escherichia coli (E. coli).

4. The method of claim 1, wherein the one or more source genes comprise a perchlorate reductase A gene (pcrA), a perchlorate reductase B gene (pcrB), a perchlorate reductase C gene (pcrC), a perchlorate reductase D gene (pcrD), and a chlorite dismutase gene (cld).

5. The method of claim 1, wherein determining the second sequence of DNA which, if inserted into the target bacteria, would allow the target bacteria to code for the ordered set of amino acids comprises:
determining a first codon frequency for one or more codons which code for amino acids in the ordered set of amino acids in the source bacteria;
determining a second codon frequency for the one or more codons in the target bacteria; and
modifying the first sequence of DNA by replacing one or more codons which code for the amino acids in the ordered set of amino acids with replacement codons which code for the amino acids but have a higher codon frequency in the target bacteria, wherein a resulting sequence comprises the second sequence of DNA.

6. The method of claim 1, further comprising applying a growth medium for the modified bacteria to the substrate.

7. The method of claim 1, further comprising, responsive to a concentration of a targeted compound in the substrate falling below a predetermined threshold, purging the substrate of the modified bacteria.

8. The method of claim 7, wherein the targeted compound comprises one of perchlorate, chlorate, or chlorite.

9. The method of claim 7, further comprising periodically or continuously sampling the substrate to determine a current concentration of the targeted compound.

10. The method of claim 7, further comprising growing a culture of the modified bacteria.

11. A non-naturally occurring Escherichia coli comprising:
a gene package comprising a sequence of deoxyribonucleic acid (DNA) that codes for an ordered set of amino acids, the ordered set of amino acids having a linear sequence that codes for a protein which breaks down perchlorates;
wherein the gene package is derived by:
determining a source bacteria comprising one or more source genes which code for perchlorate reduction and chlorite dismutase;
identifying a first sequence of DNA in the one or more source genes that codes, in the source bacteria, for the ordered set of amino acids;
determining a second sequence of DNA which, if inserted into naturally occurring Escherichia coli, would allow a resulting non-naturally occurring Escherichia coli to code for the ordered set of amino acids;
building the gene package by replacing the first sequence of DNA in the one or more source genes with the second sequence of DNA; and
inserting the gene package into naturally occurring Escherichia coli, thereby forming a non-naturally occurring Escherichia coli.

12. The non-naturally occurring Escherichia coli of claim 11, wherein the source bacteria comprises a bacteria of the genus Dechloromonas.

13. The non-naturally occurring Escherichia coli of claim 11, wherein the source bacteria comprises a bacteria of the genus Dechlorosoma.

14. The non-naturally occurring Escherichia coli of claim 11, wherein the source bacteria comprises a bacteria of the genus Azospira.

15. The non-naturally occurring Escherichia coli of claim 11, wherein the one or more source genes comprise a perchlorate reductase A gene (pcrA).

16. The non-naturally occurring Escherichia coli of claim 11, wherein the one or more source genes comprise a perchlorate reductase B gene (pcrB).

17. The non-naturally occurring Escherichia coli of claim 11, wherein the one or more source genes comprise a perchlorate reductase C gene (pcrC).

18. The non-naturally occurring Escherichia coli of claim 11, wherein the one or more source genes comprise a perchlorate reductase D gene (pcrD).

19. The non-naturally occurring Escherichia coli of claim 11, wherein the one or more source genes comprise a chlorite dismutase gene (cld).

20. The non-naturally occurring Escherichia coli of claim 11, wherein determining the second sequence of DNA which, if inserted into naturally occurring Escherichia coli, would allow a resulting non-naturally occurring Escherichia coli to code for the ordered set of amino acids comprises:
determining a first codon frequency for one or more codons which code for amino acids in the ordered set of amino acids in the source bacteria;
determining a second codon frequency for the one or more codons in a target bacteria; and
modifying the first sequence of DNA by replacing one or more codons which code for the amino acids in the ordered set of amino acids with replacement codons which code for the amino acids but have a higher codon frequency in the target bacteria, wherein a resulting sequence comprises the second sequence of DNA.
